(19) United States
(12) Patent Application Publication
(10) Pub. No.: US 2024/0175819 A1
(43) Pub. Date: May 30, 2024

(54) DIAGNOSTIC TEST

(71) Applicant: THE UNIVERSITY OF WARWICK, Coventry (GB)

(72) Inventors: Andrea DSOUZA, Coventry (GB); Jérôme CHARMET, Coventry (GB); Chrystala CONSTANTINIDOU, Coventry (GB); Rachel Alice HAND, Coventry (GB)

(21) Appl. No.: 18/072,219

(22) Filed: Nov. 30, 2022

(51) Int. Cl. G01N 21/78; C12Q 1/04; G01N 21/64

(52) U.S. Cl. CPC G01N 21/78; C12Q 1/04; G01N 21/6408

(57) ABSTRACT

A biosensor detects bacteria in a sample and/or carries out antimicrobial susceptibility testing of the bacteria to an antibiotic. The biosensor includes a crosslinked hydrogel. The crosslinked hydrogel contains a composition suspended therein, including (i) a bacterial nutrient media; (ii) optionally, an antibiotic; and (iii) an indicator for determining bacterial growth.

This invention relates generally to a diagnostic test for diagnosing and informing the treatment of an infection, e.g. a urinary tract infection (UTI), using a patient sample, e.g. a urine sample. More specifically, although not exclusively, this invention relates to a diagnostic test, preferably a rapid diagnostic test. The invention preferably relates to a diagnostic test comprising a hydrogel biosensor, methods of making the same, and methods of using the same.

Urinary tract infections (UTIs) are common bacterial infections affecting men and women of all ages, although the rate of incidence is higher in women and older age groups. According to the UKs National Institute for Health and Care Research (NIHR)—see Point-of-care testing for urinary tract infections; Horizon Scan Report 0045, June 2016, UTIs are a significant cause of mortality, especially amongst the elderly population, with UTI-related symptoms accounting for between 1-3% of all primary care consultations and being the main reason for 13.7% of community antibiotic prescriptions.

The NICE Guidance published in October 2018 (NG109) encourages an initial prescription of first-line antibiotics (e.g. trimethoprim) before urine culture for women. A urine culture to determine the nature of the bacterial infection is only performed prior to prescribing for men and pregnant women.

In clinics, the gold standard microbiology techniques involving urine culture and microbroth dilution are performed to detect and confirm the causative bacterial species. In addition, evaluation of antibiotic susceptibility is performed by disc diffusion assay to determine the treatment regimen. These tests have significant limitations including prolonged time of bacterial detection due to ˜24 to 72 hour turnaround time of bacterial growth, and extensive and laborious microbiology procedures. Due to this, as is advised by NICE, antibiotics are prescribed to some patients even before the infection status is known.

If the first-line antibiotic does not work to treat the infection, then further investigation is required. However, antibiotic susceptibility testing for resistant infections can take up to 5 days with gold standard methods, such as disc diffusion on a solid agar plate along with the test antibiotic, or broth dilution (Giuliano, C.; Patel, C. R.; Kale-Pradhan, P. B., A Guide to Bacterial Culture Identification And Results Interpretation. P t 2019, 44 (4), 192-200).

Similar treatment of UTIs worldwide has led to concerns of its contribution to antimicrobial resistance (AMR) due to inappropriate and/or over-prescription of antibiotics. It is known that the widely used first-line antibiotics such as nitrofurantoin, trimethoprim, and fosfomycin are gradually becoming ineffective for treatment of UTIs.

It is of general interest to reduce antibiotic use so as to mitigate antibiotic resistance.

UTIs are typically, but not always, caused by Escherichia coli or Klebsiella pneumoniae. It remains a challenge to identify the microbe(s) responsible for the infection, and its susceptibility to one or more antibiotics.

Point-of-care diagnostics such as dip-stick assays have drawbacks such as poor specificity and inaccurate results, often with false positives and negatives.

Automated instruments such as the VITEK® 2 offer rapid microbial identification and antimicrobial susceptibility testing, for example in approximately 5 hours. However, these instruments are expensive and require state-of-the-art facilities.

Genotypic methods involving nucleotide amplification such as polymerase chain reaction (PCR), quantitative real-time PCR, isothermal nucleic acid amplification, and sequencing-based methods have also been widely employed. However, these methods require expensive reagents and are limited to known molecular probes for bacterial detection based on existing nucleotide sequences, thus, limiting their applicability for screening a wide range of bacterial pathogens.

There are also recently emerging technologies including microfluidics, optical, and electrochemical-based platforms however, typically these methods are too complex for clinical implementation and research is still on-going.

Therefore, the diagnosis of, and the identification of appropriate and effective treatment for, UTIs remains a challenge. There is a clear need for a simple diagnostic test to determine effective treatment, e.g. by identifying the bacterial species causing the infection and/or determining its susceptibility to specific antibiotics. This targeted approach would be useful in mitigating the problem of antimicrobial resistance (AMR).

It would also be useful to be able to rapidly detect UTIs and determine an effective treatment in remote locations, e.g. in low-to-middle income countries, in a cost-effective manner.

It is therefore a first non-exclusive object of the invention to provide a diagnostic test for infections, e.g. UTIs, which mitigates or solves at least one of the aforementioned problems.

Accordingly, a first aspect of the invention provides a biosensor for detecting bacteria in a sample and/or for carrying out antimicrobial susceptibility testing of said bacteria to an antibiotic, the biosensor comprising a crosslinked hydrogel, the crosslinked hydrogel containing a composition suspended therein comprising:
i. a bacterial nutrient media;
ii. optionally, an antibiotic;
iii. an indicator for determining bacterial growth.

In embodiments, the sample may be a urine sample.

In embodiments, the bacterial nutrient media is a Mueller-Hinton broth II, e.g. a composition containing calcium and magnesium ions with beef extract (e.g. 3.0 g), casein hydrolysate (e.g. 17.5 g), starch (e.g. 1.5 g), agar (17.0 g), water (1 litre), and pH adjusted to neutral at 25° C. Mueller-Hinton broth II is commercially available from Sigma Aldrich® having the product number 90922.

In embodiments, the bacterial nutrient media is a Mueller-Hinton broth I, e.g. a composition containing beef infusion solids (2.0 g/L), casein hydrolysate (17.5 g/L), starch (1.5 g/L). Mueller-Hinton broth I does not contain calcium or magnesium ions. Mueller-Hinton broth I is commercially available from Sigma Aldrich® having product number 70192.

In embodiments, the bacterial nutrient media is Luria Bertani broth, e.g. containing Tryptone (10 g/L), Yeast Extract (5.0 g/L), and NaCl (5.0 g/L).

In embodiments, the antibiotic may be a first line antibiotic or a second line antibiotic. First line antibiotics include ampicillin, amoxicillin/clavulanic acid, cefpodoxime, cephalexin, gentamicin, trimethoprim, nitrofurantoin, mecillinam, and fosfomycin. Second line antibiotics include piperacillin/tazobactam, meropenem, ertapenem, ciprofloxacin, and amikacin. In embodiments, the antibiotic may be selected from one or more of ampicillin, amoxicillin/clavulanic acid, cefpodoxime, cephalexin, gentamicin, trimethoprim, nitrofurantoin, mecillinam, fosfomycin, piperacillin/tazobactam, meropenem, ertapenem, ciprofloxacin, and amikacin.

In embodiments, the antibiotic may be present at a minimum inhibitory concentration (MIC) of that particular antibiotic. In embodiments, the antibiotic may be present in a concentration above the minimum inhibitory concentration (MIC) of that particular antibiotic. The skilled person understands that the MIC is not a constant value because bacterial resistance changes over time. By MIC, we mean a value that is decided by a consortium such as the European Committee on Antimicrobial Susceptibility Testing, or the CLSI (Clinical and Laboratory Standards Institute).

In embodiments, the indicator may be resazurin. Advantageously, resazurin is suitable for use as an indicator for bacterial growth because it is converted from a purple and weakly fluorescent compound, to a pink and fluorescent compound (i.e. resorufin) in the presence of bacterial reductases, and/or to a colourless and non-fluorescent compound (i.e. dihydroresorufin) in the presence of bacterial acidic metabolic by-products. More advantageously, the unique growth profile of a specific bacteria (e.g. determined using fluorescence emission data) may be determined from the resazurin reduction profile.

In embodiments, the indicator may be phenol red. Advantageously, phenol red is suitable for use as an indicator for bacterial growth because it exhibits colour changes at different pH values. Phenol red is a yellow colour at a pH of less than 6.8, and is a bright pink colour at a pH of greater than 8.2, and exhibits a gradual colour change in between these pH values. The colour of the biosensor may be used to determine the amount of bacterial growth.

In embodiments, the biosensor may further comprise mannitol, e.g. in 7.5% NaCl within the biosensor. Advantageously, mannitol specifically indicates the growth of pathogenic Staphylococcus aureus due to its specific ability to ferment mannitol resulting in acid production. Therefore, the inclusion of mannitol into the biosensor provides further validation of a suspected S. aureus infection or coinfection. Inclusion of mannitol with 7.5% NaCl is a differential bacterial marker. In conditions where urine is infected with a mixed population of E. coli and S. aureus, in the presence of mannitol with 7.5% NaCl, S. aureus survives while E. coli is non-viable due to high salt concentration and its inability to ferment mannitol. In embodiments wherein the indicator is resazurin, if S. aureus survives then resazurin is converted to resorufin with an associated increase in fluorescence intensity.

Advantageously, the biosensor may be used to detect the presence of bacteria within a sample and as a diagnostic test to determine the antimicrobial susceptibility to a specific antibiotic. The bacteria nutrient media enables the rapid growth of bacteria within the hydrogel, and the indicator is able to detect said bacterial growth. In embodiments wherein an antibiotic is present, the biosensor may be used to carry out antimicrobial susceptibility testing of said bacteria to an antibiotic to determine its resistance or susceptibility to said antibiotic, by determining the impact on bacterial growth in the presence of said antibiotic.

In embodiments, the crosslinked hydrogel used in the biosensor may be fabricated from a composition comprising:
i. 2-acrylamido-2-methylpropane sulfonic acid sodium salt (Na AMPS) monomer;
ii. polyethylene glycol diacrylate (PEGDA) crosslinker;
iii. a photoinitiator.

The composition may be exposed to light, e.g. UV light, to fabricate the hydrogel for use in the biosensor, e.g. by crosslinking the components.

In embodiments, the composition for fabricating the hydrogel may comprise 2-acrylamido-2-methylpropane sulfonic acid sodium salt (Na AMPS) monomer as 50 wt. % in water.

In embodiments, the composition for fabricating the hydrogel may comprise polyethylene glycol diacrylate (PEGDA) crosslinker having a molecular weight of between 500 to 600 g/mol, e.g. 575 g/mol.

In embodiments, the ratio between the Na AMPS and PEGDA in the hydrogel may be 500 to 1 moles of Na AMPS to PEDGA, for example, from 400 to 600 moles of Na AMPS to 1 mole of PEGDA, e.g. from 450 to 550 moles of Na AMPS to 1 mole of PEGDA.

In embodiments, the composition for fabricating the hydrogel may comprise one or more photoinitiator(s) present in a catalytic quantity, e.g. from 0.1 to 5 wt. % of the total composition. In embodiments, the photoinitiator may be or comprise Irgacure 1173 (CAS No:7473-98-5, 2-hydroxy-2-methylpropiophenone).

In a specific embodiment, the hydrogel for use in a biosensor is fabricated from a composition comprising 2-acrylamido-2-methylpropane sulfonic acid sodium salt (Na AMPS) monomer (50% in water, 0.10 mol), polyethylene glycol diacrylate (PEGDA) crosslinker, molecular weight 575 g/mol (0.2 mmol), and Irgacure 1173 (0.07 mol).

In embodiments, the hydrogel may be an ultra-porous hydrogel.

In embodiments, the hydrogel may be an acrylamide-based hydrogel or a methacrylate-based hydrogel.

Advantageously, the crosslinked hydrogel for use in the biosensor is suitable for encapsulating hydrophilic compounds such as antibiotics, nutrients, and indicator compounds, e.g. resazurin. More advantageously, the crosslinked hydrogel for use in the biosensor is porous, which enables bacteria to grow both on its surface and within the internal gel network, thereby exposing the bacteria to the nutrient media, and also to the antibiotics contained within the hydrogel.

A further aspect of the invention provides a method of manufacturing a biosensor according to the invention, the method comprising providing a portion of crosslinked hydrogel, and suspending a bacterial nutrient media, an indicator for determining bacterial growth, and optionally an antibiotic within the hydrogel.

A yet further aspect of the invention provides a method of using a biosensor to detect the presence of bacteria in a sample (e.g. a urine sample), the method comprising:
i. providing a biosensor according to the invention, e.g. comprising a crosslinked hydrogel, the hydrogel containing a composition suspended therein comprising a bacterial nutrient media and an indicator for determining bacterial growth;
ii. introducing a sample (e.g. a urine sample) to the biosensor;
iii. incubating the sample within the biosensor under conditions to promote bacterial growth;
iv. detecting a change in the colour and/or fluorescence of the indicator within the biosensor to detect the presence of bacteria within the sample.

The conditions to promote bacterial growth may comprise incubating the biosensor containing the sample at 37° C. (+3°) C., e.g. for a period of time from 30 minutes to 24 hours, for example 60 minutes to 24 hours.

In embodiments, step iii. of incubating the sample within the biosensor may be performed for a period of time from 30 minutes to 24 hours, e.g. up to 60 minutes, e.g. from 60 minutes to 24 hours, e.g. from 60 minutes to 120 minutes, or from 60 minutes to 3 hours, or from 18 to 24 hours.

In embodiments, detecting a change in the colour of the indicator within the biosensor may be performed using visual analysis, e.g. by the clinician, using the naked eye. In embodiments, detecting a change in the colour of the indicator within the biosensor may be performed using a camera, e.g. a smartphone camera to visualise the colour change. This is advantageous wherein the bacteria is weakly resistant to an antibiotic, and weak colour changes may be detected on a camera, e.g. a smartphone camera.

In embodiments wherein resazurin is used as an indicator for determining bacterial growth, the following colours may be visually detected:
i) Purple (resazurin) wherein there is no bacterial growth and no conversion of resazurin has occurred, e.g. wherein the bacteria is susceptible to an antibiotic within the biosensor;
ii) Pink (resorufin) wherein bacterial growth has occurred and in turn has converted resazurin to resorufin, e.g. wherein the bacteria is resistant to the antibiotic within the biosensor;
iii) Colourless (dihydroresorufin) wherein bacterial growth has occurred, and is a species which produces acidic metabolic by-products, so has converted resazurin to resorufin, which has then been further converted to dihydroresorufin, e.g. wherein the bacteria is resistant to the antibiotic within the biosensor.

In embodiments, detecting a change in the fluorescence of the indicator within the biosensor may comprise performing fluorescence spectroscopy, e.g. using a fluorimeter or a fluorescent plate reader, to record an emission spectrum or provide an emission value.

In embodiments, detecting a change in the colour and/or fluorescence of the indicator within the biosensor to detect the presence of bacteria within the sample may comprise comparing a control value, e.g. from the biosensor before a sample is introduced or, e.g. from a separate control sample, with a detected value, e.g. a visually detected colour change or a fluorescence emission signal from the biosensor.

In embodiments, detecting a change in the colour and/or fluorescence of the indicator within the biosensor to detect the presence of bacteria within the sample may comprise detecting a single end point colour change or fluorescence emission signal.

In embodiments, detecting a change in the colour and/or fluorescence of the indicator within the biosensor to detect the presence of bacteria within the sample may comprise detecting the change in colour and/or fluorescence emission over time, e.g. continuously or by taking plural discrete readings over a set period of time.

In embodiments, detecting the change in fluorescence emission over time by taking plural discrete readings over a set period of time may comprise taking from 3 to 120 readings over 60 minutes, e.g. from 5 to 100 readings, or from 10 to 80 readings, or from 20 to 60 readings, or from 30 to 40 readings over 60 minutes. In embodiments, detecting the change in fluorescence emission over time by taking plural discrete readings over a set period of time may comprise taking a reading every 1, 2, 3, 4, or 5 minutes, or every 10 minutes, or every 15 minutes, e.g. for a total time of up to 30 minutes, or up to 60 minutes.

Advantageously, when using resazurin as the indicator for determining bacterial growth, the change in fluorescence may be monitored, e.g. using plural discrete readings over a set period of time, to provide a measure of the bacterial growth (e.g. a growth curve over time) within the biosensor of the invention.

In embodiments, the method may comprise generating a growth profile of the bacteria using data on the change in fluorescence emission over time.

In embodiments, the method may also be able to identify the type (e.g. species) of bacteria in a sample (e.g. a urine sample) by further comprising the step of comparing the change in fluorescence emission of the indicator (e.g. over time) to data (e.g. previously recorded fluorescence emission data indicating the growth profile of a known bacterial species) within a database to identify the type (e.g. species) of bacteria in a sample (e.g. a urine sample).

The data within the database may comprise the growth profile of a bacteria in the form of fluorescence emission data over time.

Advantageously, any aspect of the invention may further comprise integrated machine learning to accurately determine the bacterial species.

A yet further aspect of the invention provides a method of using a biosensor to determine the resistance or susceptibility of bacteria within a sample (e.g. a urine sample) to a specific antibiotic, the method comprising:
i. providing a biosensor comprising a crosslinked hydrogel, the hydrogel containing a composition suspended therein comprising a bacterial nutrient media, an antibiotic, and an indicator for determining bacterial growth, e.g. resazurin;
ii. introducing a sample (e.g. a urine sample) to the biosensor;
iii. incubating the sample within the biosensor under conditions to promote bacterial growth;
iv. detecting a change in the colour and/or fluorescence of the indicator within the biosensor to determine the resistance or susceptibility of bacteria within the sample to an antibiotic.

The conditions to promote bacterial growth may comprise incubating the biosensor containing the sample at 37° C., e.g. for a period of time from 30 minutes to 24 hours, for example 60 minutes to 24 hours.

In embodiments, step iii. of incubating the sample within the biosensor may performed for a period of time from 30 minutes to 24 hours, e.g. up to 60 minutes, or from 60 minutes to 24 hours.

In embodiments, detecting a change in the colour of the indicator within the biosensor may be performed using visual analysis, e.g. by the clinician, using the naked eye. In embodiments, detecting a change in the colour of the indicator within the biosensor may be performed using a camera, e.g. a smartphone camera to visualise the colour change. This is advantageous wherein the bacteria is weakly resistant to an antibiotic, and weak colour changes may be detected on a camera, e.g. a smartphone camera.

In embodiments, detecting a change in the fluorescence of the indicator within the biosensor may comprise performing fluorescence spectroscopy, e.g. using a fluorimeter or a fluorescent plate reader, to record an emission spectrum or provide an emission value.

In embodiments, detecting a change in the colour and/or fluorescence of the indicator within the biosensor to determine the resistance or susceptibility of bacteria within the sample an antibiotic may comprise comparing a control value, e.g. from the biosensor before a sample is introduced, or e.g. from a separate control sample, with a detected value, e.g. visually detected colour change or a fluorescence emission signal from the biosensor.

In embodiments, detecting a change in the colour and/or fluorescence of the indicator within the biosensor may comprise detecting a single end point colour change or fluorescence emission value or signal.

In embodiments, detecting a change in the colour and/or fluorescence of the indicator within the biosensor to determine the resistance or susceptibility of bacteria within the sample to an antibiotic may comprise detecting the change in colour and/or fluorescence emission over time, e.g. continuously or by taking plural discrete readings over a set period of time.

In embodiments, the method may comprise generating a growth profile of the bacteria using data on the change in fluorescence emission over time.

In embodiments, detecting the change in fluorescence emission over time by taking plural discrete readings over a set period of time may comprise taking from 3 to 120 readings over 60 minutes, e.g. from 5 to 100 readings, or from 10 to 80 readings, or from 20 to 60 readings, or from 30 to 40 readings over 60 minutes. In embodiments, detecting the change in fluorescence emission over time by taking plural discrete readings over a set period of time may comprise taking a reading every 1, 2, 3, 4, or 5 minutes, or every 10 minutes, or every 15 minutes, e.g. for a total time of up to 30 minutes, or up to 60 minutes.

Advantageously, when using resazurin as the indicator for determining bacterial growth, the change in fluorescence may be monitored, e.g. using plural discrete readings over a set period of time, to provide a measure of the bacterial growth (e.g. a growth curve over time) within each biosensor of the device. The presence of bacterial growth indicates that the bacteria is resistant to the antibiotic within the biosensor, and the absence of bacterial growth indicates that the bacteria is susceptible to the antibiotic within the biosensor. In this way, an appropriate antibiotic may be prescribed for the treatment of the infection.

A further aspect of the invention provides a device comprising plural biosensors according to the invention.

In embodiments, each biosensor of the device comprises a different antibiotic, e.g. at or above a minimum inhibitory concentration (MIC).

In embodiments, the device comprises plural biosensors each comprising a different antibiotic, and a control biosensor without an antibiotic. The control biosensor may comprise a portion of crosslinked hydrogel, the hydrogel containing a composition suspended therein comprising a bacterial nutrient media and an indicator for determining bacterial growth, e.g. resazurin.

In embodiments, the device comprises plural biosensors, each comprising a different concentration of the same antibiotic, e.g. at and above the MIC of the antibiotic.

Advantageously, the use of plural biosensors according to the invention in a device enables testing to be multiplexed and/or run in parallel to determine which antibiotic is most effective for treating the UTI.

A yet further aspect of the invention provides a method of using the device according to the invention to determine the resistance or susceptibility of bacteria within a sample (e.g. a urine sample) to plural antibiotics, the method comprising:
i. providing plural biosensors each comprising a crosslinked hydrogel, the hydrogel containing a composition suspended therein comprising a bacterial nutrient media, an antibiotic, and an indicator for determining bacterial growth, e.g. resazurin, wherein each biosensor comprises a different antibiotic;
ii. introducing a sample (e.g. a urine sample) to each biosensor;
iii. incubating the sample within the plural biosensors under conditions to promote bacterial growth;
iv. detecting a change in the colour and/or fluorescence of the indicator within each biosensor, and comparing this to a control biosensor absent an antibiotic, to determine the resistance or susceptibility of bacteria within the sample.

Any of the previous statements of invention in relation to the method of using a biosensor to determine the resistance or susceptibility of bacteria within a sample may apply to the method of using the device of the invention to determine the resistance or susceptibility of bacteria within a sample to plural antibiotics.

In embodiments, the method of using the device according to the invention to detect the presence of bacteria in a sample, to identify the type (e.g. species) of bacteria in a sample, and/or to determine the resistance or susceptibility of bacteria within a sample to one or more antibiotics, may be performed concurrently in the same biosensor.

Advantageously, the biosensor may be used to determine the presence of an infection caused by bacteria. More advantageously, the biosensor may be used to identify the appropriate antibiotic, or combination of antibiotics, which may be used to treat the infection. Most advantageously, this is useful to mitigate or prevent the use of inappropriate antibiotics to treat infections, e.g. UTIs.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Embodiments of the invention will now be described by way of example only with reference to the accompanying drawings in which:

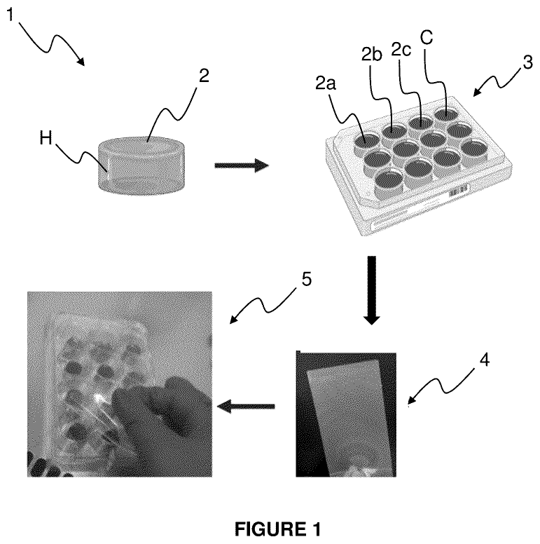

FIG. 1 is an illustration of a biosensor and a device according to the invention; and

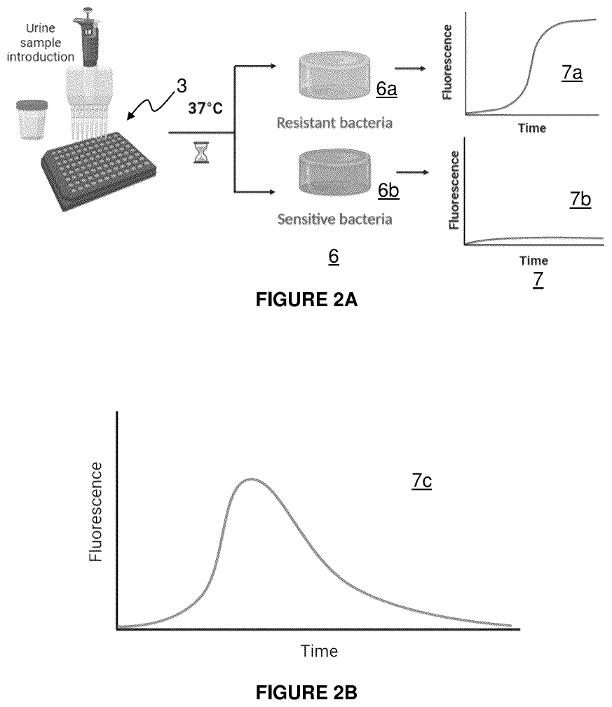

FIG. 2A is a scheme according to a method of using a biosensor to determine the resistance or susceptibility of bacteria within a urine sample to a specific antibiotic according to the invention;

FIG. 2B is a model graph showing the fluorescence emission data recorded over time for a resistant bacteria and/or viable bacteria producing acidic metabolic by-products;

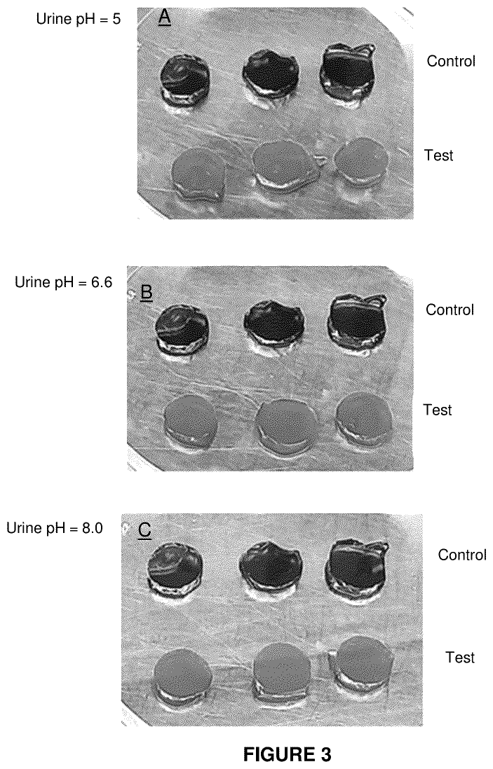

FIG. 3 is a series of photographs showing the visual colour change of biosensors in the presence of bacteria in urine of different pH values within 60 minutes, according to Examples of the invention;

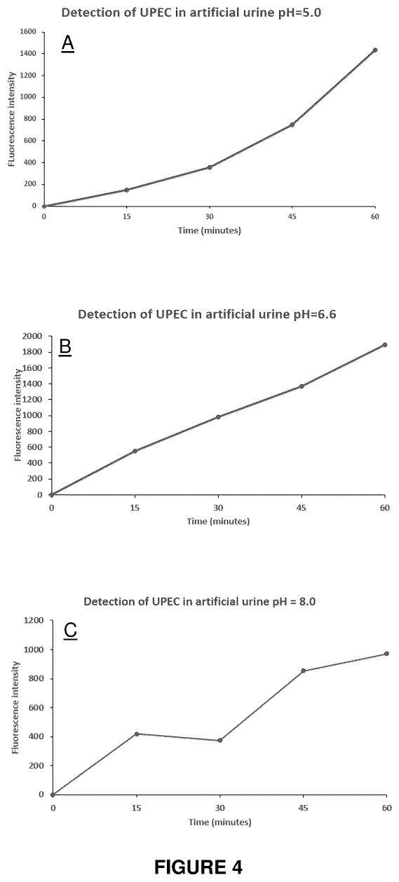

FIG. 4 is a series of graphs showing the fluorescence emission of biosensors in the presence of bacteria in urine of different pH values within 60 minutes, according to Examples of the invention;

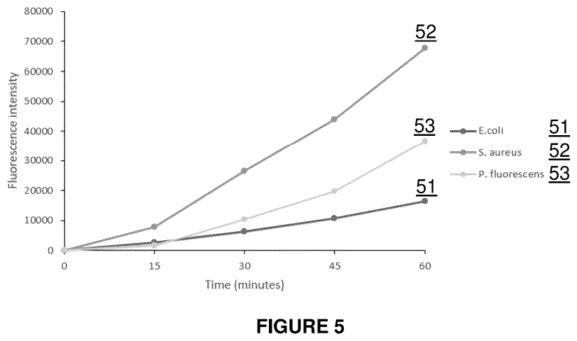

FIG. 5 is growth detection profile data of different bacteria (Escherichia coli (E. coli), Pseudomonas fluorescens (P. fluorescens), Staphylococcus aureus (S. aureus) within 60 minutes, according to Examples of the invention;

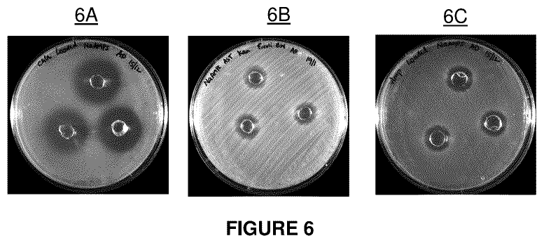

FIG. 6 is a series of photographs of biosensors each comprising a different antibiotic wherein bacterial growth is inhibited as a result of antibiotic release from the hydrogel network;

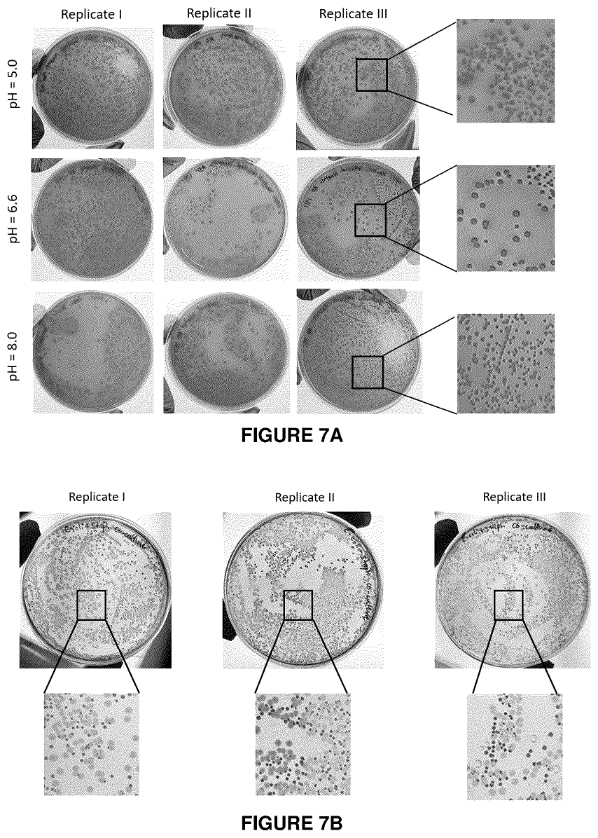

FIGS. 7A and 7B are plates showing bacterial growth of bacteria retrieved from biosensors of the invention;

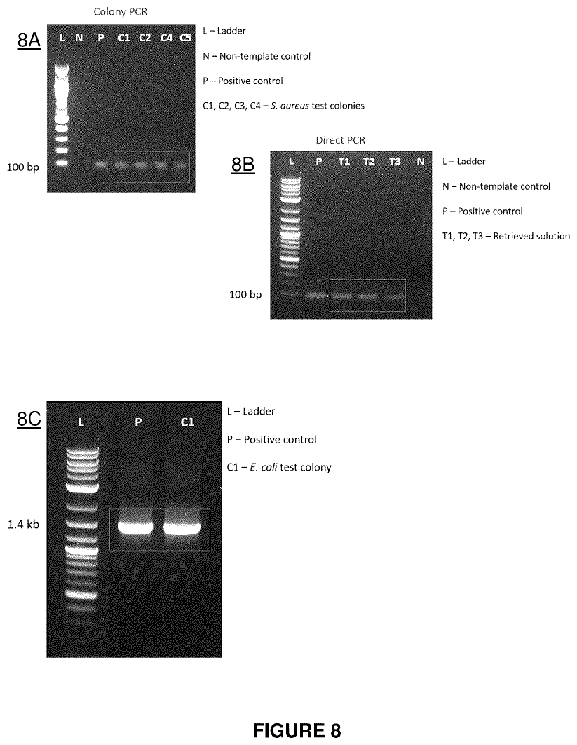

FIG. 8 shows the results of nucleic acid amplification tests of bacteria retrieved from biosensors of the invention;

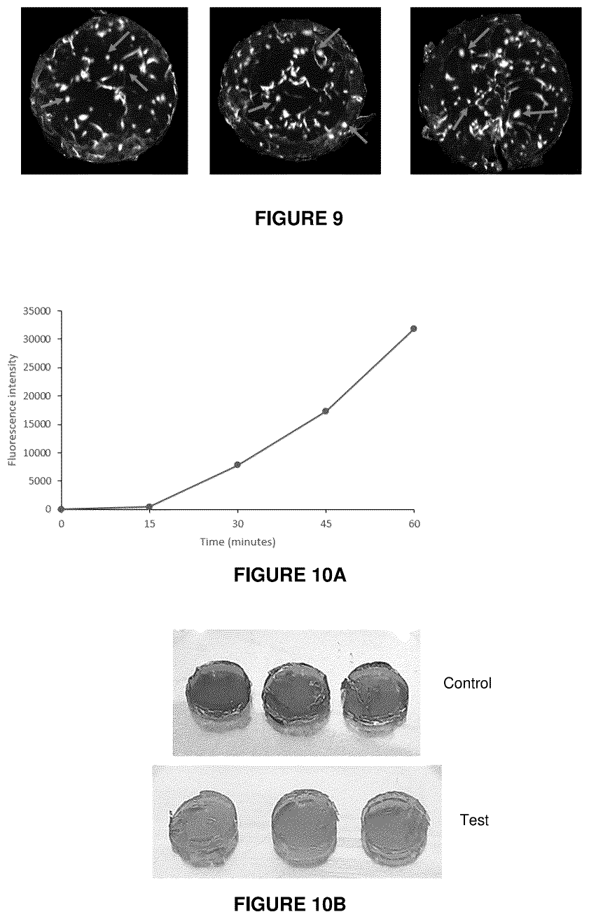

FIG. 9 is a series of images which show bacterial growth using Luria Bertani broth as the bacterial nutrient media;

FIG. 10A is a graph showing the growth profile of E. coli grown in a biosensor containing MHB1 as the bacterial nutrient media;

FIG. 10B is a series of photographs showing the visual colour change of biosensors in the presence of E. Coli wherein MHB1 is used as the bacterial nutrient media, according to Examples of the invention.

Referring now to FIG. 1, there is shown a method 1 of assembling a device 5 from plural biosensors 2, according to the invention. There is shown a biosensor 2, plural biosensors 2a, 2b, 2c . . . , and a part assembled device 3, a film 4, and a fully assembled device 5 according to the invention.

The biosensor 2 comprises a crosslinked hydrogel H. In this embodiment, the crosslinked hydrogel H was fabricated from a composition comprising 2-acrylamido-2-methylpropane sulfonic acid sodium salt (Na AMPS) monomer with 50 wt. % water, polyethylene glycol diacrylate (PEGDA) crosslinker having a molecular weight of 575 g/mol, and Irgacure 1173 (2-hydroxy-2-methylpropiophenone) as a photoinitiator. The final concentration of Na AMPS used is 0.10 mol within the hydrogel of the biosensor. The final concentration of PEGDA crosslinker is 0.2 mmol within the hydrogel of the biosensor. The hydrogel is crosslinked using UV light.

Advantageously, it has been found that the hydrogel made according to this protocol produces efficient swelling kinetics, which is essential for rapid encapsulation and release of antibiotics, and encapsulation of bacterial growth viability indicator. In addition, said hydrogel are mechanically stable that are capable of supporting bacterial growth on the surface as well as within the hydrogel network.

The hydrogel (2 mL) is crosslinked, and swollen with the bacterial growth media broth, resazurin, and/or antibiotics (wherein the control biosensor does not comprise an antibiotic). This causes the hydrogel to approximately double in size and weight. The hydrogel portions are then cut to a specific size after they have been swollen with the media solution, ˜1.8 cm diameter with ˜0.7 cm thickness. This volume is introduced into the biosensor, e.g. a well in a plate. Other volumes of hydrogel may also be used, e.g. starting with 1 mL of hydrogel before swelling.

The crosslinked hydrogel H of the biosensor 2 contains a composition suspended therein comprising:
i. a bacterial nutrient media;
ii. an antibiotic;
iii. an indicator for determining bacterial growth.

The composition is introduced to the crosslinked hydrogel H and is suspended throughout the network via a hydrogel swelling mechanism.

In this embodiment, the indicator for determining bacterial growth is resazurin.

The part-assembled device 3 comprises plural biosensors 2a, 2b, 2c . . . , according to the invention, each containing a different antibiotic. The biosensor “C” is the control, which is fabricated in the same way as for the biosensors 2a, 2b, 2c . . . but absent an antibiotic. The biosensors 2a, 2b, 2c . . . , C, may be pre-deposited or introduced into the part-assembled device 3. In this embodiment, each biosensor 2a, 2b, 2c . . . comprises a different antibiotic.

The part-assembled device 3 may be sealed, for example with a transparent self-adhesive film 4 to protect from moisture and the external environment. The seal 4 may be removed or pierced to introduce a sample, e.g. of urine (not shown).

In this embodiment, the device 5 is a multi-well plate compatible with a fluorescence plate reader for real-time fluorescence emission readings. For example, the multi-well plate may have 12, 24, or 96 wells to enable multiplexed testing.

Ideally, the device 5 is stored at a temperature of 4° C. for use at a later date.

Referring now to FIG. 2A, there is shown a scheme according to an embodiment of a method of using a biosensor to determine the resistance or susceptibility of bacteria within a urine sample to a specific antibiotic according to the invention. There is shown a device 3 according to the invention wherein the film 4 has been removed. The device 3 comprises plural biosensors (not labelled).

There is also shown the results 6, 7 of the method of the invention. There is shown a biosensor 6a comprising a urine sample wherein the bacteria were resistant to the antibiotic, and a biosensor 6b comprising a urine sample wherein the bacteria were sensitive to the antibiotic. There is also shown a graph 7a of fluorescence emission over time, wherein the bacteria were resistant to the antibiotic within the biosensor, and a graph 7b of fluorescence emission over time, wherein the bacteria were sensitive to the antibiotic within the biosensor.

In a method of the invention, a user or clinician introduces ˜an aliquot of a urine sample (e.g. 20 ul) into each biosensor 2a, 2b, 2c (shown in FIG. 1, not labelled in FIG. 2A). A sample is introduced into a control biosensor C (i.e. absent an antibiotic) and each biosensor 2a, 2b, 2c comprising a different antibiotic.

Upon sample introduction, the device 3 is incubated (37° C., 60 minutes) to provide favourable conditions for bacterial growth. The porous nature of the hydrogel enables any bacteria present in the urine sample to adhere to the hydrogel surface and also permeate into the hydrogel network to access the nutrient media encapsulated within the hydrogel.

Referring first to biosensor 6a, wherein the bacteria are resistant towards the specific antibiotic within the biosensor. In this case, the bacteria will use the nutrients to replicate. The bacterial growth causes an increase in the quantity of reductase enzyme within the hydrogel network. The reductase enzyme reacts with the resazurin indicator to reduce it to resorufin. This causes the hydrogel to change in colour from purple and weakly fluorescent (resazurin) to pink and fluorescent (resorufin). The biosensor 6a may be visually inspected to determine the colour change from purple (resazurin) to pink (resorufin), indicating bacterial growth.

In other cases (not shown), certain bacteria produce acidic by-products during their metabolism, which causes further oxidation of resorufin resulting in the formation of dihydroresorufin. Dihydroresorufin is colourless and is characterized by a drastic drop in fluorescence intensity. The biosensor may also be visually inspected to determine the colour change from purple (resazurin) to colourless (dihydroresorufin) indicating bacterial growth of a species producing acidic metabolic by-products.

Referring first to biosensor 6b, in the case wherein the bacteria are susceptible to the specific antibiotic within the biosensor, then the bacteria is prevented from replicating. Therefore, less or no reductase enzyme is generated, so less or no reaction occurs with the resazurin in the hydrogel, and there is less or no colour change to the biosensor 6b which remains purple (resazurin). Visual inspection may also be used to determine the lack of colour change.

Referring now to the graphs 7a and 7b, the fluorescence emission signal over time of each biosensor 6a, 6b is detected. This data is used to determine the extent of bacterial growth, and thus determine whether the bacteria in the sample are susceptible or resistant to the antibiotic within the biosensor.

The fluorescence emission data recorded over time in the graph 7a for the biosensor 6a containing the resistant bacteria produces a sigmoidal curve with increasing fluorescence emission over time.

In contrast, biosensor 6b contains the sensitive bacteria and as such, there is no increase in the fluorescence signal because the resazurin is not converted to resorufin.

In the case wherein the biosensor (not shown) contains resistant bacteria which produce acidic metabolic by-products, the resazurin is converted to resorufin, which is in turn converted to dihydroresorufin. Referring also to FIG. 2B, there is shown a graph 7c showing the fluorescence emission data recorded over time having a bell curve wherein the fluorescence signal increases over time as resazurin is converted to resorufin, and then the fluorescence signal decreases over time as the resorufin is converted to dihydroresorufin.

Advantageously, the conversion of resazurin to resorufin and/or dihydroresorufin is an indication of bacterial growth. More advantageously, each bacteria exhibits a unique growth profile based on the conversion of resazurin to resorufin and the associated change in fluorescence, which may be detected, e.g. using plural discrete readings, over time. Therefore, based on resazurin conversion curves, the specific bacterial species causing the infection may be identified. More advantageously, poly-bacterial infections may be identified based on the unique mixed growth profiles.

Most advantageously, the device according to the invention is able to simultaneously identify the bacterial species responsible for the infection and determine which antibiotic is most appropriate for treatment.

The method of the invention may be automated, e.g. using a robot to carry out one or more of the steps of the method.

The multiplexed device 3 can either be used with multiple patient samples and/or to determine the susceptibility to plural antibiotics by using plural biosensors, e.g. 2a, 2b, 2c.

### EXAMPLES

The invention is now be exemplified with the following Examples.

Referring now to FIG. 3, there is shown a series of biosensors comprising resazurin without an antibiotic, and confirming the presence of uropathogenic E. coli in artificial urine at different pH conditions, according to Examples of the invention. There is shown three of each biosensor assay comprising (A) urine pH 5.0; (B) urine pH 6.6; and (C) urine pH 8. There are also three controls shown for each pH value.

The presence of uropathogenic E. coli is confirmed with the colour change from purple (resazurin) to pink (resorufin), which may be visually inspected using the naked eye.

Referring now to FIG. 4, there is shown a series of graphs illustrating fluorescence emission over time (hours) of the biosensors used in the assays of FIG. 3, and spiked with uropathogenic E. coli in artificial urine (initial load of 10⁴ CFU/ml) of different pH values. The increasing fluorescence intensity over time resulting from the conversion of resazurin to resorufin confirms the presence of the bacteria in the urine sample. There is shown fluorescence intensity over time graphs for biosensors wherein the pH of the urine sample introduced was: (A) 5.0; (B) 6.6; and (C) 8.0.

The results of FIGS. 3 and 4 demonstrates that the biosensor assay functions at different pH values, which is useful because infected urine has a broad pH range.

Referring now to FIG. 5, there is shown a graph showing growth detection profile data of different bacteria (Escherichia coli (E. coli) (line 51), Pseudomonas fluorescens (P. fluorescens) (line 53), Staphylococcus aureus (S. aureus) (line 52) within 60 minutes, according to Examples of the invention. All species were present within the sample at 105 CFU/mL.

It is shown that the method of the invention enables both qualitative detection due to the visible colour change (shown in the photographs), and quantitative detection by detecting the fluorescence emissions to provide unique bacterial growth profiles.

Advantageously, the hydrogel of the biosensor may be retrieved, after the test has been run, for use in techniques to characterise the pathogenic bacteria responsible for the infection. For example, said techniques may include phenotypic assays such as bacterial plating and typing, and genotypic assays such as nucleic acid amplification-based and sequencing methods. This type of analysis may be used to characterise pathogenic bacteria during outbreaks and to identify the emergence of new bacterial infections.

In an specific example, the hydrogel was retrieved from the biosensor after the method of the invention was performed. The retrieved hydrogel was suspended in 10 ml MHB (Mueller-Hinton Broth II) followed by orbital shaking (180 RPM at 37° C.). The presence of the bacterial species E. coli and S. aureus was confirmed by bacterial plating including MacConkey agar and CLED-agar (phenotypic assays) and 16S rRNA PCR (genotypic assay) in two separate examples.

Referring now to FIG. 6, there is shown photographs of three biosensors 6A, 6B, 6C each comprising a different antibiotic encapsulated within the crosslinked hydrogel network. Biosensor 6A comprises the antibiotic chloramphenicol, biosensor 6B comprises the antibiotic kanamycin, and biosensor 6C comprises the antibiotic ampicillin. The biosensors 6A, 6B, 6C were spiked with E. coli. In each case, it is shown that there is antibacterial activity in the area surrounding the spiked portion of hydrogel. Therefore, in these cases, E. coli. is shown to be susceptible to the antibiotics encapsulated within the biosensors 6A, 6B, 6C.

Referring now to FIGS. 7A and 7B, there is shown results indicating successful retrieval of pathogenic bacteria from artificial urine specimens in a biosensor of the invention after the assay has been performed. FIG. 7A shows growth of E. coli. on MacConkey agar, which selectively and differentially identifies Gram-negative bacterial species due to their ability to ferment lactose. The colonies indicate the growth of uropathogenic E. coli after retrieval from the biosensor of the invention. FIG. 7B shows growth of retrieved mixed population of bacteria including uropathogenic E. coli and Staphylococcus aureus after their detection on biosensor of the invention. In this experiment, CLED (Cysteine, Lactose, Electrolyte-Deficient) agar was used to differentiate mixed bacterial species. The brown colonies indicate E. coli growth while opaque yellow colonies indicate S. aureus growth.

Referring now to FIG. 8, there is shown nucleic acid amplification tests 8A, 8B, 8C of bacteria retrieved from a biosensor of the invention after the assay has been performed. In the results of 8A, bacteria retrieved from biosensor were grown on Luria Bertani Broth plate to produce colonies followed by colony PCR. Colony PCR indicates S. aureus amplified product of size 100 bp, confirming its retrieval from the biosensor. In the results of 8B, PCR was performed directly on retrieved S. aureus from biosensor (without colony formation). The direct PCR also confirms the retrieval of S. aureus from biosensor. In the results of 8C, colony PCR of retrieved E. coli from the biosensor confirms its successful recovery.

Referring now to FIG. 9, there is shown a series of images which show bacterial growth using Luria Bertani broth as the bacterial nutrient media. The images show the growth of fluorescent E. coli colonies on the biosensor encapsulated with Luria Bertani broth. This demonstrates that the Na AMPS hydrogel may comprise Luria Bertani broth as the bacterial nutrient media to support bacterial growth. This is a qualitative indication of bacterial growth in which the bacteria itself is fluorescent in nature and is grown on the biosensor. The images are taken at a wavelength of 488 nm. The bright spots indicate bacterial fluorescence as indicated in red to represent a few colonies.

Referring now to FIG. 10A, there is shown a graph showing the growth profile of E. coli grown in a biosensor containing MHB1 (Mueller Hinton Broth 1) as the bacterial nutrient media and resazurin as the indicator for bacterial growth. The graph shows quantitative detection of the bacteria within the biosensor wherein the fluorescence intensity increases as resazurin is converted to resorufin. FIG. 10B is a series of photographs showing the visual colour change of biosensors used in FIG. 10A. The photographs show qualitative detection of the bacteria within the biosensor wherein the colour changes from purple to pink to indicate bacterial growth.

Advantageously, this shows that different bacterial nutrient media may be used in the biosensor.

Advantageously, the invention enables simultaneous bacterial detection and antibiotic susceptibility in less than an hour, eliminates the need for sample preparation, e.g. urine culture, bacterial extraction, and other extensive microbiology procedures; and provides both qualitative as well as quantitative detection.

Advantageously, the biosensor and device of the invention is rapid (e.g. complete within one hour), cost-effective and with a low limit of detection (10 CFU/mL) plus with minimal urine sample preparation.

It will also be appreciated by those skilled in the art that any number of combinations of the aforementioned features and/or those shown in the appended drawings provide clear advantages over the prior art and are therefore within the scope of the invention described herein.

1. A biosensor for detecting bacteria in a sample and/or for carrying out antimicrobial susceptibility testing of said bacteria to an antibiotic, the biosensor comprising a crosslinked hydrogel, the crosslinked hydrogel containing a composition suspended therein comprising:
i. a bacterial nutrient media;
ii. optionally, an antibiotic;
iii. an indicator for determining bacterial growth.

2. A biosensor according to claim 1, wherein the bacterial nutrient media is selected from a Mueller-Hinton broth I, Mueller-Hinton broth II, a Luria Bertani broth or combinations of two or three of said broths.

3. A biosensor according to claim 1, wherein the antibiotic is present and is selected from one or more of ampicillin, amoxicillin/clavulanic acid, cefpodoxime, cephalexin, trimethoprim, gentamicin, nitrofurantoin, mecillinam, fosfomycin, piperacillin/tazobactam, meropenem, ertapenem, ciprofloxacin, and amikacin.

4. A biosensor according to claim 1, wherein the indicator for determining bacterial growth is selected from resazurin or phenol red.

5. A biosensor according to claim 1, wherein the crosslinked hydrogel is fabricated from a composition containing 2-acrylamido-2-methylpropane sulfonic acid sodium salt (Na AMPS) monomer and polyethylene glycol diacrylate (PEGDA) crosslinker.

6. A method of using a biosensor to detect the presence of bacteria in a sample, the method comprising:
i. providing a biosensor according to the invention, e.g. comprising a crosslinked hydrogel, the hydrogel containing a composition suspended therein comprising a bacterial nutrient media and an indicator for determining bacterial growth;
ii. introducing a sample to the biosensor;
iii. incubating the sample within the biosensor under conditions to promote bacterial growth;
iv. detecting a change in the colour and/or fluorescence of the indicator within the biosensor to detect the presence of bacteria within the sample.

7. A method according to claim 6, wherein the indicator for determining bacterial growth is resazurin or phenol red.

8. A method according to claim 6, wherein the conditions to promote bacterial growth comprise incubating the biosensor containing the sample at 37° C.

9. A method according to claim 6, wherein step iii. of incubating the sample within the biosensor is performed for a period of time from 30 minutes to 24 hours.

10. A method according to claim 6, wherein step iv. of detecting a change in the colour of the indicator within the biosensor is performed by visual analysis using the naked eye or using a camera.

11. A method according to claim 6, wherein step iv. of detecting a change in the fluorescence of the indicator within the biosensor comprises taking plural discrete readings of the fluorescence emission of the biosensor over a set period of time.

12. A method according to claim 11, wherein the set period of time is about 60 minutes.

13. A method according to claim 12, wherein the fluorescence emission data over time is used to identify the type or species of bacteria within the sample by comparing with previously recorded data for known bacterial species.

14. A method of using a biosensor to determine the resistance or susceptibility of bacteria within a sample to a specific antibiotic, the method comprising:
i. providing a biosensor comprising a crosslinked hydrogel, the hydrogel containing a composition suspended therein comprising a bacterial nutrient media, an antibiotic, and an indicator for determining bacterial growth;
ii. introducing a sample to the biosensor;
iii. incubating the sample within the biosensor under conditions to promote bacterial growth;
iv. detecting a change in the colour and/or fluorescence of the indicator within the biosensor to determine the resistance or susceptibility of bacteria within the sample to an antibiotic.

15. A method according to claim 14, wherein the indicator for determining bacterial growth is selected from resazurin or phenol red.

16. A method according to claim 14, wherein the conditions to promote bacterial growth comprise incubating the biosensor containing the sample at 37° C.

17. A method according to claim 14, wherein step iii. of incubating the sample within the biosensor is performed for a period of time from 30 minutes to 24 hours.

18. A method according to claim 14, wherein step iv. of detecting a change in the colour of the indicator within the biosensor is performed by visual analysis using the naked eye or using a camera.

19. A method according to claim 14, wherein step iv. of detecting a change in the fluorescence of the indicator within the biosensor comprises taking plural discrete readings of the fluorescence emission of the biosensor over a set period of time.

20. A method according to claim 20, wherein the same is a urine sample.
